(19) United States
(12) Patent Application Publication
(10) Pub. No.: US 2008/0234627 A1
(43) Pub. Date: Sep. 25, 2008

(54) PIVOTALLY ENGAGED MULTIPLE PART ELECTROTRANSPORT DRUG DELIVERY DEVICE

(76) Inventors: Wanda Dent, Chanhassen, MN (US); Johannes N. Gaston, Minnetonka, MN (US); Douglas J. Vanornum, Menasha, WI (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/052,517

(22) Filed: Mar. 20, 2008

Related U.S. Application Data
(60) Provisional application No. 60/896,396, filed on Mar. 22, 2007.

(51) Int. Cl. A61N 1/30

(52) U.S. Cl. 604/20

(57) ABSTRACT

An electrotransport device for delivering a therapeutic agent through a body surface of a patient. The device has an electronic module that can be coupled with an agent module to form the electrotransport device. The agent module has a compartment containing the therapeutic agent for delivery through the body surface by electrotransport. The agent module has a first end, and includes first agent module (AM) coupler about the first end. The electronic module has a first end and a second end corresponding to the first end and second end of the agent module. The electronic module has a first electronic module (EM) coupler about the first end of the electronic module for coupling with the first AM coupler such that as the first AM coupler matingly engages with the first EM coupler the electronic module and the agent module can be pressed together pivoting about where the first AM coupler engages the first EM coupler. The electronic module includes circuitry for electrically driving the therapeutic agent for electrotransport.

### CROSS REFERENCE TO RELATED U.S. APPLICATION DATA

The present application is derived from and claims priority to provisional applications U.S. Ser. No. 60/896,396, filed Mar. 22, 2007, which is herein incorporated by reference in its entirety.

### TECHNICAL FIELD

The present invention relates to a multiple part electrotransport drug delivery system for driving ionic drug across a body surface or membrane. In particular, the invention relates to a system having an electronic part and a drug reservoir part that can be coupled together before drug delivery.

### BACKGROUND

The delivery of active pharmaceutical agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

The natural barrier function of the body surface, such as skin, presents a challenge to delivery therapeutics into circulation. Devices have been invented to provide transdermal delivery of drugs. Transdermal drug delivery can generally be considered to belong to one of two groups: transport by a “passive” mechanism or by an “active” transport mechanism. In the former, such as fentanyl transdermal systems available from Jassen Pharmaceuticals and other drug delivery skin patches, the drug is incorporated in a solid matrix, a reservoir with rate-controlling membrane, and/or an adhesive system.

Passive transdermal drug delivery offers many advantages, such as ease of use, little or no pain at use, disposability, good control of drug delivery and avoidance of hepatic first-pass metabolism. However, many active agents are not suitable for passive transdermal delivery because of their size, ionic charge characteristics, and hydrophilicity. Most passive transdermal delivery systems are not capable of delivering drugs under a specific profile, such as by ‘on-off’ mode, pulsatile mode, etc. Consequently, a number of alternatives have been proposed where the flux of the drug(s) is driven by various forms of energy. Some examples include the use of iontophoresis, ultrasound, electroporation, heat and microneedles. These are considered to be “active” delivery systems.

One method for transdermal delivery of such active agents involves the use of electrical current to actively transport the active agent into the body through intact skin by electrotransport. Electrotransport techniques may include iontophoresis, electroosmosis, and electroporation. Electrotransport devices, such as iontophoretic devices are known in the art, see, e.g., U.S. Pat. Nos. 5,057,072, 5,084,008, 5,147,297, 6,039,977, 6,049,733, 6,171,294, 6,181,963, 6,216,033, and US Patent Publication 20030191946. One electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the substance to be driven into the body is ionic and is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

A prior iontophoretic system similar to that of U.S. Pat. No. 6,181,963 is shown in FIG. 1. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable polymer.

Printed circuit board assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23, 23′ in the depressions 25, 25′ formed in lower housing, by means of electrically conductive adhesive strips 42, 42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′, 29 in adhesive 30. The skin-facing side 36 of the adhesive 30 has adequate adhesive property to maintain the device on the skin for the duration of the use of the device.

Recently, there have been suggestions to provide different parts of an electrotransport system separately and connect them together for use. For example, such connected-together systems might provide advantages for reusable controller circuit. In reusable systems, the drug-containing units are disconnected from the controller when the drug becomes depleted and a fresh drug-containing unit is then connected to the controller again. Examples of electrotransport devices having parts being connected together before use include those described in U.S. Pat. No. 5,320,597 (Sage, Jr. et al), U.S. Pat. No. 4,731,926 (Sibalis), U.S. Pat. No. 5,358,483 (Sibalis), U.S. Pat. No. 5,135,479 (Sibalis et al.), UK Patent Publication GB2239803 (Devane et al), U.S. Pat. No. 5,919,155 (Lattin et al.), U.S. Pat. No. 5,445,609 (Lattin et al.); U.S. Pat. No. 5,603,693 (Frenkel et al.), and WO1996036394 (Lattin et al.).

However, many of the prior connected-together systems are cumbersome to use and do not provide for easy assembly and use.

What is needed is an electrotransport device in which the electronic part and the reservoir part can be easily assembled about the time of use.

### SUMMARY

The present invention relates to an electrotransport device for delivering a therapeutic agent through a body surface of a patient. The device has an electronic module that can be coupled with an agent module (AM) to form the electrotransport device before use. The present invention provides such electrotransport devices and methods of making and using such electrotransport devices. In one aspect, the agent module has a compartment (e.g., reservoir) containing the therapeutic agent for delivery through the body surface by electrotransport. The agent module has a first agent module (AM) coupler about a first end and the electronic module has a first electronic module (EM) coupler about the same end for coupling with the first AM coupler such that as the first AM coupler matingly engages with the first EM coupler the electronic module and the agent module can be pressed together pivoting about where the first AM coupler engages the first EM coupler. The electronic module has circuitry for electrically driving the therapeutic agent for electrotransport. Because of the shapes of (AM) coupler and the corresponding (EM) coupler the two modules can be easily oriented to match fit together.

In an aspect, the present invention provides a method of making an electrotransport device for delivering a therapeutic agent through a body surface of a patient. The method includes matingly engaging an insert at one end of one of an agent module and an electronic module to a receptor at the other of said modules and pressing the modules together by pivoting about where the modules engage. The agent module contains a compartment (e.g., reservoir) including the therapeutic agent and the electronic module includes circuitry for controlling electrotransport. The present invention also provide electrotransport devices and methods of making electrotransport devices wherein the method including matingly engaging a tongue having a curve or angled portion about a first end of one of an agent module and an electronic module to a receptor at the other of said modules and pressing the modules together by pivoting about where the modules matingly engage. The agent module contains a compartment including the therapeutic agent and the electronic module includes circuitry for controlling electrotransport.

In another aspect, the present invention provides an electrotransport device and a method of making an electrotransport device for delivering a therapeutic agent through a body surface of a patient wherein an insert at one end of one of an agent module and an electronic module can engage with a receptor at the other of said modules allowing the modules to freely pivot toward or away from each other. The modules can be pressed together by pivoting about where the modules engage. The agent module contains a compartment including the therapeutic agent and the electronic module includes circuitry for controlling electrotransport.

In another aspect, an electrotransport device and a method of making are provided in which the device has an agent module that can be coupled with an electronic module, which is multilayered. In one aspect, the electronic module has an upper cover and a lower cover sandwiching or surrounding about and protecting a printed circuit board, which contains circuitry for electrically driving the therapeutic agent for electrotransport. One of the layers in the electronic module has a coupler that couples with a coupler in the agent module to provide pivotal motion for the modules to be affixed together. The multilayered construction of the electronic module, and preferably of the agent module allows for appropriate placement of the couplers to facilitate pivotal movement. Because the layers can be made separately and then affixed together, either by mechanical anchoring, chemical bonding or by molding together by heat, the coupler such as tongues and feet with openings, can be made at strategic locations for optimal pivotal movement. Typically only relatively small couplers (e.g., a small tongue and a corresponding small receptor) are needed for providing pivotal movement (compared to the size of the electronic module and the reservoir module). In certain embodiments, the layered construction of the electronic module and the reservoir module provides advantages in making and positioning of the couplers. The layered construction further provides protection of the electronics from mechanical disturbance and moisture as well as protection of the reservoir(s) from mechanical disturbance.

The present invention also provides methods of making and methods of using the above electrotransport devices. After the electronic module has been coupled to the agent module, the device can then be applied onto the body surface of a patient. The present invention provides module designs that make them easily oriented and aligned by identifying the couplers through inspection of the ends of the modules. An insert can be matched with a receptor to ensure correct assembly with correct polarity match. Further the sliding motion for first engaging the modules at one end of the device accompanied by pivoting motion provides a natural fluid motion for bringing the modules together for full assembly. Because of the wrist of a person is adapted for a pivotal motion itself, the curve or bend construction in certain embodiment of the insert in a module further facilitates insertion of the insert into a receptor for initially coupling one end of the modules. After engaging one end, the pivotal motion allows a lever action that facilitates the final engagement at the end distal from the pivotal fulcrum. Thus, the present invention provides devices that can be easily assembled. It is to be understood that the present invention of engaging two modules can be applied to electrotransport devices such as iontophoretic devices electroosmosis devices, and electroporation devices, as long as there are two modules that need to be coupled together for mechanical and electrical engagement.

### BRIEF DESCRIPTION OF THE FIGURES

The present invention is illustrated by way of examples in embodiments and not limitation in the figures of the accompanying drawings in which like references indicate similar elements. The figures are not shown to scale unless indicated otherwise in the content.

FIG. 1 illustrates an exploded perspective view of a prior art typical electrotransport system.

FIG. 2 illustrates an exploded perspective view of an embodiment of an electrotransport system of the present invention.

FIG. 3A illustrates a perspective view of an embodiment of a lower cover in an electronic module of an electrotransport system of the present invention.

FIG. 3B and FIG. 3C are perspective views of an embodiment of portion of an inner upper portion in a reservoir module of an electrotransport system of the present invention.

FIG. 4A and FIG. 4B are schematic side view representations of an embodiment showing in portion the lower cover of the electronic module and the inner upper portion of the reservoir module being assembled.

FIG. 5A to FIG. 5B are schematic side view representations of embodiments showing in portion the lower cover.

FIG. 5C to FIG. 5D are schematic side view representations of embodiments showing in portion the lower cover and in portion of the inner upper portion.

FIG. 6A shows a top plan view of the embodiment of FIG. 2 assembled.

FIG. 6B shows a perspective top view of an embodiment of a device of the present invention with a digital display.

FIG. 7A and FIG. 7B show schematic side view representations of embodiments of the lower covers of the electronic modules of electrotransport systems of the present invention.

FIG. 8A and FIG. 8B show schematic side view representations of embodiments of electrotransport systems of the present invention with further variations in the couplers that provide pivotal movement and coupling.

FIG. 9 shows a schematic perspective view of an embodiment of an electrical connector (coupler receptor) for an electrotransport system of the present invention.

FIG. 10 shows a schematic perspective top view of an embodiment of another electrical connector for an electrotransport system of the present invention.

FIG. 11 shows a schematic perspective bottom view of the embodiment of the electrical connector of FIG. 10.

### DETAILED DESCRIPTION

The present invention is directed to an electrotransport drug delivery system that has two parts that are assembled together before drug administration to a patient. In particular, the system includes an agent-containing module (“agent module” for short) (AM) having a compartment (e.g., reservoir) containing the drug (or therapeutic agent) and an electronic module for coupling to the agent module (e.g., reservoir module) to drive the drug in electrotransport through a body surface.

The practice of the present invention will employ, unless otherwise indicated, conventional methods used by those skilled in the art of mechanical and electrical connections in drug device development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers. As used herein “matingly engaging” means an inserting coupler is inserted into a receptive coupler to a substantially full extent, e.g., by pressing the couplers firmly together before pivoting begins.

### MODES OF CARRYING OUT THE INVENTION

The present invention provides an electrotransport device that is assembled before use for electrotransport delivery of ionic compounds (e.g., ionic drugs such as fentanyl and analogs, polypeptides, protein, and the like) through a body surface, such as skin.

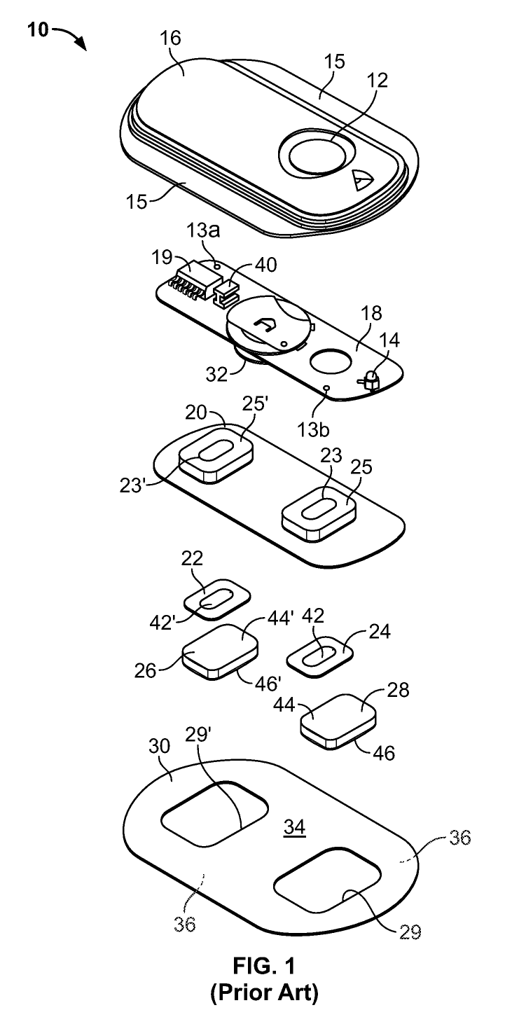

Electrotransport devices, such as iontophoretic devices are known in the art, e.g., U.S. Pat. No. 6,216,033. The structures, drugs, and electrical features of U.S. Pat. No. 6,216,033 and in FIG. 1 can be adapted to equivalents to be used in the present invention, as can be understood by one skilled in the art. In an iontophoretic drug delivery device, there is a drug reservoir and a counter reservoir.

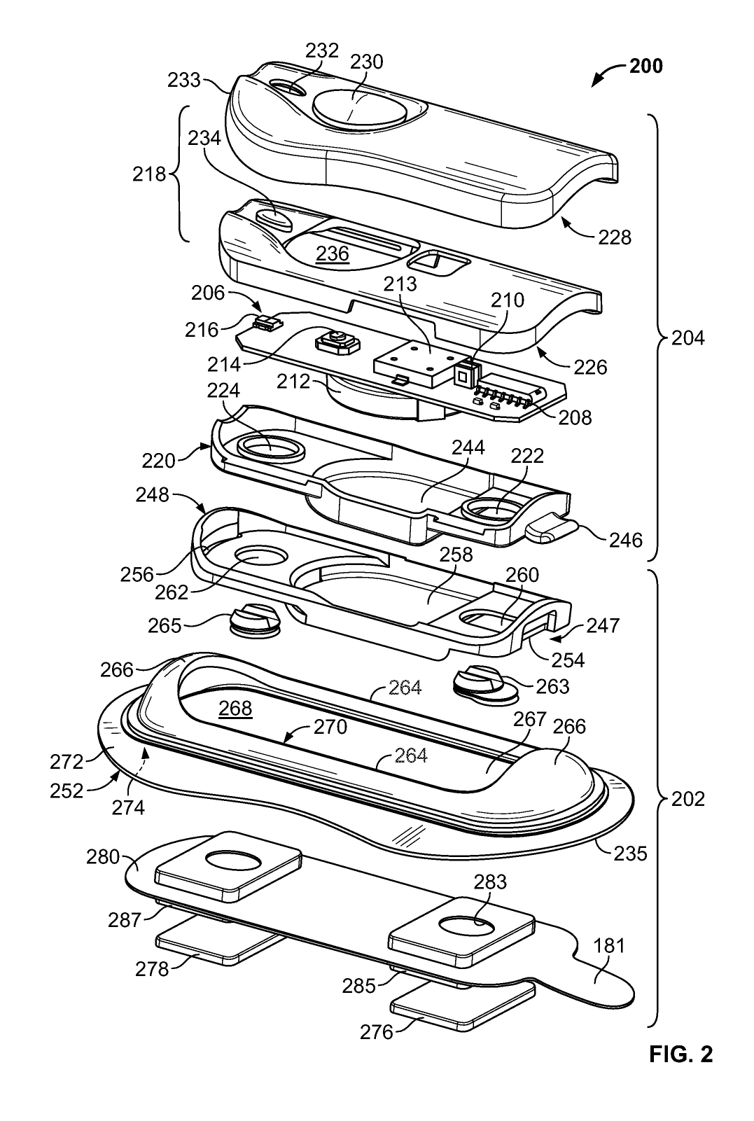

FIG. 2 shows an embodiment of an electrotransport device of the present invention. The electrotransport device 200 includes an agent module (which is a reservoir module in this embodiment) 202 and an electronic module 204. The electronic module 204 includes printed circuit board (PCB) assembly 206 that includes an integrated circuit 208 coupled to discrete electrical components 210 and power source (e.g., battery) 212. A switch 214 is connected to the integrated circuit 208 for together controlling the operation of the electrotransport device 200. An optical display (e.g., LED) 216 acts as an indicator for the operation of the device 200 to show functions of the device 200, e.g., indicating whether the device is in a drug delivery mode, the amount of drug that has been delivered up to the moment, the number of doses the device has been activated to deliver, etc. An alternative display would be a digital or alpha-numeric display (e.g., liquid crystal display) for showing the operation functions and parameters of the device. Information that can be displayed includes the number of doses already delivered. Examples of electronic components that can be present include audible alarm (shown as transducer 213) to alert the user of undesirable conditions, successful initiation of dose delivery, etc., and other features that improve or support the functions of the integrated circuit, display, etc.

The PCB assembly 206 is sandwiched between a top cover (or upper cover) 218 and a bottom cover (or lower cover) 220 so that the PCB assembly 206 is enclosed and protected by them except for electrical connectors (not shown in FIG. 2 because it is hidden in the perspective view) that are positioned at openings (holes) 222 and 224 of the lower cover 220 for electrically connecting to corresponding connectors in the reservoir module 202. Mechanical connection and engagement that secure or lock the connectors together can also be present.

The upper cover 218 of the electronic module 204 includes a lower layer 226 made of rigid or semirigid material (e.g., polypropylene) and an upper layer 228 made of a less rigid elastomer, such as ethylene vinyl acetate or ethylene-octene copolymer, e.g., ethylene-octene copolymers available under the tradename ENGAGE® from Dow Chemical Company. The polymeric material from which the upper layer 228 is made is softer and more resilient than the polymeric material of the lower layer 226 so that when the electronic module is coupled to match with the reservoir module 202 the upper layer 228 can match contours snuggly with the reservoir module to provide a splash-water resistant or liquid resistant (drip-proof) seam, as well as provide visual confirmation of correct assembly. In other words, liquid will not penetrate to cause failure of the device through the liquid resistant seam during occasional momentary water exposure such as splashing as under a short spray. Further, by using materials that are hydrophobic and/or that can butt tightly, the seam can be made to keep out aqueous liquid such as water in normal daily routine use. For example, the material at the seam can have a coating of a hydrophobic material such as polytetrafluoroethylene. A button cover portion 230 of the upper layer 228 is adequately flexible and soft such that finger pressure by a finger pressing on the button cover portion 230 can activate or deactivate the switch 214. It is noted that other elastomers or semi-rigid polymers can also be used so long as it provides an adequate amount of resiliency.

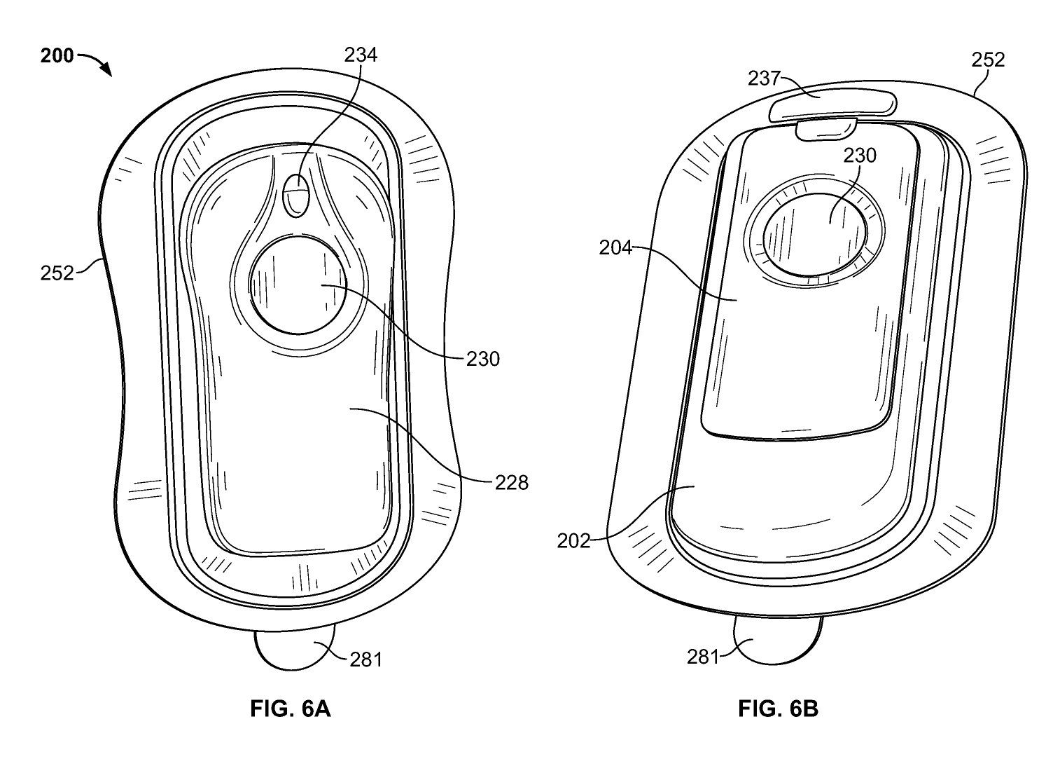

A cutout 232 on the upper layer 228 at the anterior end 233 of the device 200 allows light transmitted from the display 216 to be visible from the top view. Of course, in alternative designs, the display itself can also include LED, digital display, etc. for displaying information. For example, the embodiment in FIG. 6B has a digital display 237. For the sake of convenience of illustration, in this and similar embodiments, the end having the display 216 is considered the “anterior” end and the direction toward the upper layer 228 from the reservoir module 202 is considered “top” and “upper” whereas the direction towards the reservoir module 202 from the upper layer 228 is considered “bottom” or “lower” herein. The end opposite to the anterior end 233 is considered the posterior end 235 and a line traversing from the anterior end to the posterior end is considered to traverse longitudinally.

In this embodiment, the lower layer 226 of the upper cover 218 of the electronic module 204 is made of a transparent or translucent polymeric material that is stiffer than the upper layer 228 to protect the PCB assembly 206. A window portion 234 fits into the cutout 232 in the upper layer 228 and allows light emitted from the display 216 to be seen through the transparent or translucent window portion 234 from a top view. A cutout (opening) 236 on the lower layer 226 above the display 216 allows the display 216 to be viewed through the transparent or translucent window portion 234. The cutout 236 can alternatively be covered by a transparent or translucent material allowing the display 216 to be seen. Useful transparent or translucent polymeric material for the window 234 includes acrylic, polycarbonate, polyethylene, polypropylene, polyethylene terephthalate, and the like. Further, glass fibers, glass particles, silica, and the like can also be included in the transparent or translucent polymeric material to provide more stiffness, to provide support and protection of the PCB assembly 206 and to secure to the lower cover 220 of the electronic module 204. Additives to enhance the bonding in the polymeric materials and dispersion aids to improve dispersion of additives or light in the transparent or translucent material can also be used in the molding material. For a digital display, a window with sufficient transparency for the digits to be read is provided.

The lower cover 220 of the electronic module 204 has a cavity 244 for accommodating the battery 212 and has openings 222 and 224 allowing electrical connectors from the PCB assembly 206 to electrically connect with the reservoir module 202. The lower cover 220 further has couplers 246, 257, which can couple with receptor couplers 247, 256 respectively from the reservoir module 202. (Coupler 257 is not visible from FIG. 2 because it is hidden in the perspective view.) Alternatively, the openings 222 and 224 instead of being open voids rather can be conductive pads that are electrically and mechanically connected to the electronics in the PCB. The conductive pads allow the electrical connectors from the reservoir module 202 to connect electrically to the electronic module 204.

The reservoir module 202 is typically a disposable unit that can be discarded after use with appropriate procedure. The reservoir module 202 has a rigid inner upper portion 248 and has a less rigid outer upper portion 252 surrounding the more rigid inner upper portion 248 laterally and at the anterior end 233 and the posterior end 235. The inner upper portion has a generally layer shape. At one end of the device, the inner upper portion 248 has coupler receptor 247 having an opening 254 for receiving the tongue of the coupler 246 of the electronic module 204. At another end of the device, another receptor 256 with opening(s) is there to lockingly receive another coupler insert(s) (not shown because it is hidden in the perspective view) extending from the lower cover 220 of the electronic module 204. The couplers at the two ends 233, 235 can have different structures for securing the two modules together. The couplers at the two ends can both have a single insert and receptor hole, or two or more inserts and receptor holes, or one end can have inserts and receptor holes different from the other end. Inserts and receptor holes can be located at either the electronic module side or the reservoir module side. For the coupling with a tongue for pivotal movement, e.g., coupler 246, it is preferred that the tongue be on the coupler of the electronic module 204.

A cavity 258 in the inner upper portion 248 provides for space to accommodate the portion of the lower cover 220 protecting the battery 212. Openings 260, 262 securely accommodate electrical connectors 263, 265 that provide electrical connection between the electrical connectors of the electronic module 204 and the electrode current distributor 285, 287. The electrical connectors 263, 265 have grooves or other securing features for securing them to the inner upper portion 248 at the openings 260, 262. The openings 260, 262 can have rims around them to fit into the grooves of the electrical connectors 263, 265 for interference fit for securing together, or for ensuring good electrical connection by digging into the material of electrical connectors 263, 265. The electrical connectors 263, 265 can be made of metal or carbonized polymer to make them conductive. Alternatively, the electrical connectors 263, 265 can be comolded with the inner upper portion 248.

The inner upper portion 248 in the reservoir module 202 and the lower cover 220 in the electronic module 204 can be made with relatively stiff material, preferably electrically insulating polymeric material so that they can be coupled together to provide a sturdy support structure for the PCB assembly 206 and the flange (or wing) 272 of the outer upper portion 252 in the reservoir module 202. A layer of adhesive can be provided under the flat flange (or wing) 272 for attachment to the body surface. Useful material for making the inner upper portion 248 and the lower cover 220 include polyethylene, polypropylene, polyethylene terephthalate, polystyrene, and the like. Glass fibers, glass particles, silica, and the like can also be included in the polymeric material to provide more stiffness. When two materials are molded together, they are selected so that they are compatible for comolding, e.g., having similar thermal and chemical property. Further, pigments and other material can also be included in the construction material for the pieces that provide mechanical support. The stiff material also provides a means to create secure mechanical attachment that may be independent of the electrical connection.

The outer upper portion 252 in the reservoir module 202 includes a cutout 268 for receiving and securing the inner upper portion 248. Means for securing the various portions and pieces together can include couplers such as mating insert and receptors, adhesive, frictionally interfering edges, etc. Towards the posterior end 235, the outer upper portion 252 includes an upwardly extending side ridges 264 and end ridges 266. The ridges 264, 266 edge a cutout 268 having a channel 267 through which the lower cover 220 in the electronic module 204 can be received. As the electronic module 204 is installed with the reservoir module 202, the side ridges 264 guide the side edges of the lower cover 220 into the channel 267.

The outer upper portion 252 in the reservoir module 202 has a thin, generally flat annular flange 272 extending from the frame 270 all around to provide a lower surface 274 under the flat annular flange 272 for adhesive attachment to a body surface (e.g., human skin) when the device 200 is applied to the patient. Adhesive is not shown in FIG. 2 for the sake of clarity of the figure. Other than an annular shape, the thin flat flange can have the form of wings on the lateral sides of the outer upper portion 252 (similar to wings 15 shown in FIG. 1). The thin flat flange 272, because of its flexibility, can conform to the contour of the skin surface. As used herein, “annular” means ring-shaped, whether it be exactly circular, off-circular, oval, oblong, or shaped like a race track in a stadium. The outer upper portion 252 is made of a relatively soft, pliable, resilient material such as an elastomer, e.g., ethylene octene copolymer, silicone, butyl rubber, etc. Preferably the material is a biocompatible polymer (, e.g., ethylene octene copolymer (see US patent publication US20020128591) that does not absorb the beneficial agent or interact with the adhesive or other material in the reservoir, such as the matrix material of the hydrogel. The ridges 264, 266 surround the cutout 268. The ridges 264, 266 rise above the flat flange 272 to form a frame 270 to receive the corresponding portions of the electronic module. The frame 270 has an inner perimeter to receive the inner upper portion 248 such that when the inner upper portion 248 of the reservoir module 202 is fitted into the outer upper portion 252, the inner upper portion can accommodate the lower cover 220 of the electronic module 204. When the electronic module 204 and the reservoir module 202 are secured together, preferably, the upper layer 228 in the electronic module 204 can press on the frame 270 to provide a seam that is splash water resistant (or liquid resistant).

In this embodiment, the reservoir module 202 has reservoirs (preferably hydrogel) 276, 278 on the under side of the reservoir module 202 for contacting body surface of a patient for electrotransport of ions. A lower layer 280 in the reservoir module 202 is located at and secured to the underside of the upper inner portion 248. The lower layer 280 has downwardly facing cavities 283 for accommodating current distributors 285, 287 and reservoirs 276, 278. If desired, a tab 281 can extend off one end (e.g., posterior end 235) of the lower layer 280 in the reservoir module 202. An authorized person (such as a medical worker, e.g., doctor or nurse) can grasp the tab 281 to pull off the lower layer 280 with the reservoirs 276, 278 from the reservoir module 202 for disposal after the prescribed electrotransport delivery by the device 200 is completed. In this way, the risk for drug abuse through illicit use of the device is reduced.

Although it is possible to include electronic components in the reservoir module, to reduce the complexity of the reservoir module, reduce the risk of electronics failing because of corrosion due to the presence of liquid and moisture, and result in easier manufacturing processes, it is preferred that the reservoir module contains no active electronic components such as transistor, integrated circuit, operational amplifier, etc. Active electronic components are those that can provide gain in an electrical circuit, such as transistors, field effect transistors, triodes, etc. Preferably the only electrical components present in the reservoir module are nonactive components. In some embodiments, the only electrical material present in the reservoir module is conductor leading to the electrode that connect to a reservoir.

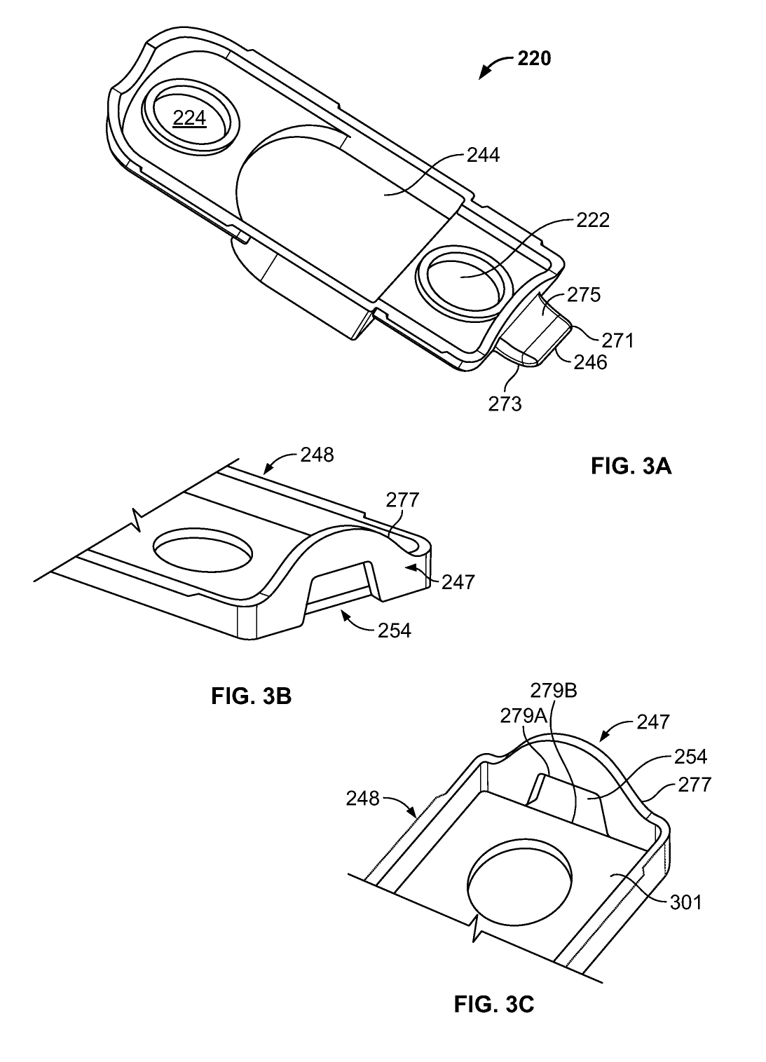

FIG. 3A shows a perspective view of the lower cover 220 in the electronic module 204. The coupler 246 has tongue 271 extending generally in the posterior direction. The tongue 271 has a curved portion 273 having the concave face 275 facing upward, i.e., away from the direction of the reservoir module 202.

FIG. 3B and FIG. 3C show the posterior portion of the inner upper portion 248 of the reservoir module 202. The inner upper portion 248 at its posterior end has coupler 247 having a foot 277 extending as a bend upward from the generally anterior-posterior orientation of the inner upper portion 248. The opening 254 is located at the bottom portion of the foot 277 where the foot is bent from the inner upper portion 248 to accommodate the tongue 271. Although the opening 254 can be made to be big enough for the tongue 271 to fit in with a lot of room for freedom of movement, preferably the opening is just large enough for the tongue 271 to fit in such that as the tongue 271 is matingly inserted into the opening 254 the concave face 275 slides on the top edge 279A of the opening 254 or the convex face slides on the bottom edge 279B or the upward face 301. A user can also position the tongue 271 and the foot 277 such that the concave face 275 slides on the top edge 279A and the convex face of the tongue 271 slides against the inner upper portion 248. It is noted that the top edge or bottom edge of the opening can have optional gaps and need not be continuous. In a preferred embodiment, the foot 277 is short (i.e., only a little taller than the height of the opening 254) such that it is not in the way of the tongue to allow extensive pivotal movement of the electronic module relative to the reservoir module. Preferably with the tongue engaging the foot 277, the two modules can pivot about an angle of 30° or more, more preferably 45° or more, even more preferably 75° or more. With a curved tongue, even an angle of 90° or more can be achieved. The foot 277 can further extend at an off-vertical angle allowing an obtuse angle with the main part of the inner upper portion 248. In this way, an even larger angle of pivotal movement between the two modules can be achieved, e.g., 100° or more, or even 135° or more. Allowing larger pivotal angles will make engaging and coupling the two modules easier.

Because of the curvature of the curved face, the sliding motion of the tongue 271 against the inner upper portion 248 at the opening 254 results a sliding rocking motion. Preferably the opening 254 is a rectangular slot and the tongue 271 has a generally rectangular cross section so that the tongue is confined to travel substantially only in a direction that follows along the length of the tongue as it traverses through the slot and that during the pivoting motion the two modules move in a book-like action. As used herein, the term “rock” or “rocking” means a movement in which a generally convex surface appears to be in contact (or actually contact) with a surface that has less curvature (e.g., a flat surface) and the convex surface appears to move in relation to as it appears to roll on the surface with less curvature. On the other hand, the concave surface of the tongue can contact the inner upper portion 248 on the other side of the opening 254, i.e. against edge 279A to provide a similar rocking appearance. In this “rocking” movement, the structure with the convex surface appears to pivot or tilt as a rocking chair appears to pivot or tilt in its rocking motion. In the preferred way of inserting the tongue 271 into the opening 254, there is no back and forth rocking. The initial engagement of the tongue 271 with the opening 254 at the foot 277 provides a fulcrum for pivotal movement to bring the anterior ends of the electronic module 204 and the reservoir module 202 as the two modules are being coupled together.

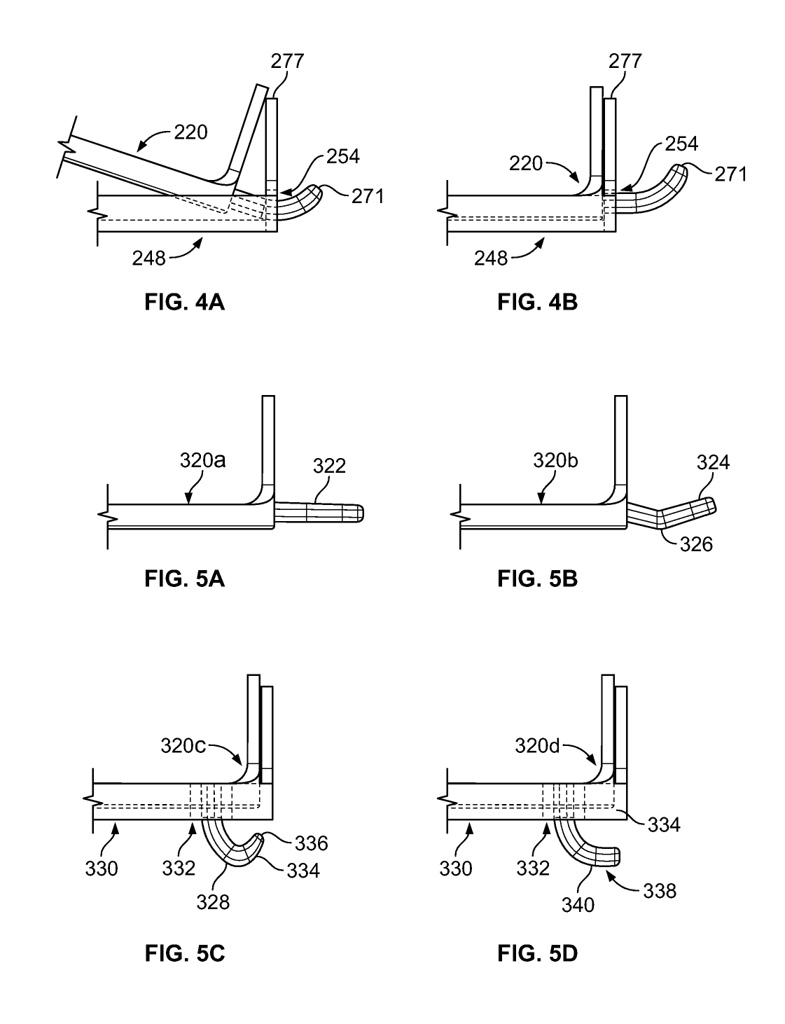

FIG. 4A shows a schematic side view in portion of the posterior portion of the lower cover 220 and the posterior portion of the inner upper portion 248 in the reservoir module 202 as the tongue 271 is in the process of being inserted through the opening 254. In this process, as the electronic module 204 is being coupled with the reservoir module 202 through the pivotal engagement at one end, the ridges 264, 266 in the outer upper portion 252 guide the side edges of the lower cover 220 into the channel 267. This way, once one end is engaged, the assembly can be finished even without visual inspection, if desired. In fact, with the presence of the tongue, the ends of the two modules can be easily detected by tactile feel and the whole inspection can be done without visual inspection if desired.

FIG. 4B shows a schematic side view in portion of the posterior portion of the lower cover 220 and the posterior portion of the inner upper portion 248 in the reservoir module 202 after the tongue 271 has been inserted fully through the opening 254. Before the tongue 271 is fully inserted into the opening 254, the inner upper portion 248 and the lower cover 220 are free to move in a pivoting motion and there is preferably no biasing force forcing them toward or away from each other. This is different from a situation in which one end of the electronic module is locked with one end of the reservoir module (e.g., by means of a snap-fit button/socket lock) wherein the locking causes a biasing force that tends to bring the other ends of the two modules closer together. The snap-fit lock can be releasable. For example, a springy catch can engage, e.g., a groove in the other module. The springy catch can be pulled to release the snap-fit lock.

FIG. 5A shows another embodiment of a lower cover 320A having a substantially straight tongue 322 extending in a substantially posterior direction along the longitudinal direction of the lower cover. FIG. 5B shows another embodiment of a lower cover 320B having a tongue 324 extending as a whole in a substantially posterior direction along the longitudinal direction of the lower cover. The tongue 324 has a bend 326 positioned between two substantially straight portions to facilitate insertion into the opening 254 at the inner upper portion 248. In the above embodiments, the tongue after full insertion into the opening is constrained from moving in the up/down and lateral directions about the opening by the foot 277. Further, after the tongue is fully inserted, the inner upper portion 248 cannot move in the posterior direction because it is blocked by the foot 277. The only direction the foot 277 allows the inner upper portion 248 to move is the anterior direction. However, when the electronic module is fully coupled to the reservoir module, couplers at the anterior end (i.e., the end opposite to the tongue) hold the lower cover 220 from moving longitudinally relative the inner upper portion 248. Thus, after the electronic module 204 and the reservoir module are coupled together at both the anterior and posterior ends, the two modules are held together securely. The tongues in the above embodiments have the advantage that after the lower cover 220 has been inserted fully into the opening 254, as long as couplers at the anterior ends of the electronic module 204 and the reservoir module 202 are not yet engaged, the modules can freely pivot and the tongue can be disengaged from the foot readily and freely if desired by simply performing a sliding motion.

FIG. 5C shows another embodiment of a lower cover 320C having a curved tongue 328 extending in a substantially downward direction perpendicular to the longitudinal direction of the inner upper portion 330 of the reservoir module 202. An opening 332 is provided on the flat body of the inner upper portion 330. The curved tongue 328 is shown inserted through opening 332. To prevent the tongue 328 from falling out through the opening 332 after assembling the electronic module and the reservoir module together, a bend 334 provides a finger extension 336 to form a hook shaped configuration at the end of the tongue. The finger extension 336 can have a point extending out to catch against the bottom side of the inner upper portion 330. Because the finger extension 336 points in a direction that is substantially non-perpendicular to the plane of the inner upper portion 330, but rather in a direction that has a significant vector component along the longitudinal direction, it prevents the lower cover 320C from disengaging from the inner upper portion 330 by arresting motion in the up direction. The bend 334 can in fact be at an angle, e.g., an acute angle, so that after full insertion through the opening 332 the finger extension 336 is directed (i.e., pointed) not only at a posterior direction, but also has a vector component at an upward direction, i.e., pointing back at the foot 227 to lock the tongue 332 with the inner upper portion 330. The size and direction of the bend of finger extension 336 can be such that once the tongue is firmly pressed through the opening 332 the tongue 328 is permanently locked from retreating through the opening 332 again. It is to be understood that the tongue 328 can in other embodiments have other features such as a bend, (with or without a curve other than the bend), the bend having a directional components that prevent upward movement. For example, FIG. 5D shows an embodiment in which the lower cover 320D has a tongue 338 that includes bends 332, 340, which are bent at obtuse angles. The obtuse angled bends allow the tongue to be freely slid in and out of the opening 332 with a generally rolling motion. The tongue in FIG. 5D is also freely disengageable from the opening at the foot even after full insertion, if desired.

With the embodiment shown in FIG. 5C, which the tongue 328 can loosely engage with the inner upper portion 330 of the reservoir module 202 (e.g., the insert can rest gently on the receptor) to still allow free pivotal motion. By “loosely engaging”, it is meant that the tongue has not been pressed so firmed that the hook has been fully pushed through the opening 332 that it becomes locked by the inner upper portion 330 and cannot be separated again easily. Thereafter, the other end (e.g., the anterior end 233) of the device can be engaged. With the posterior end 235 loosely engaged, the electronic module 204 and the reservoir module 202 are pressed together at the anterior end 233. Locking couplers at the anterior end (i.e., the end opposite from the end with the tongue in FIG. 2) can be pressed together to lock the modules together firmly. About the end distal (or opposite) from the disengageable tongue, the couplers can be made with hooks, buttons, barbs, angled ledges, and the like to provide a click-fit or snap-fit to make the engagement permanent or at least hard to disengage when the electronic module 204 and the reservoir module 202 are firmly pressed together (i.e., one piece can be inserted into another with a snap or click as a resilient portion of a piece is squeezed initially by a restriction and suddenly released from the squeeze once the portion has passed the restriction). One kind of insert that can also be used is bar-shaped or has a cross section of a slot U that can be press-fitted to insert into a channel-shaped receptor. In certain designs, the coupler can be designed so that it is not reengageable. For example, the insert can have a barb or hook that if it is pulled back after insertion into a receptor the barb or hook will rip or distort the receptor so that if reinserted into the receptor the receptor will no longer hold or retain the insert. Alternatively, the barb or hook can be designed to break if pulled so that it cannot reengage with the receptor. Such designs may be used to prevent illicit reuse the device or the reservoir module.

Preferably the tongue is oriented at a direction that has a directional vector component pointing to the posterior and extends past the opening in the posterior direction after full assembly. The vector representing a bent or curved tongue is taken to be the straight line joining the tip and the base of the tongue. In this way, the tongue (e.g., tongue 271) cannot be pulled away from the opening (e.g., opening 254) of the inner upper portion (e.g., 248) by a separating force perpendicular from the plane of the lower cover (e.g., 220) or the inner upper portion (e.g., 248) after full assembly. More preferably the tongue is oriented at a direction that has a directional vector having a major component pointing to the posterior and extend past the opening. As used herein the term major component when applied to vector means it is larger than any other vector component of the resultant vector.

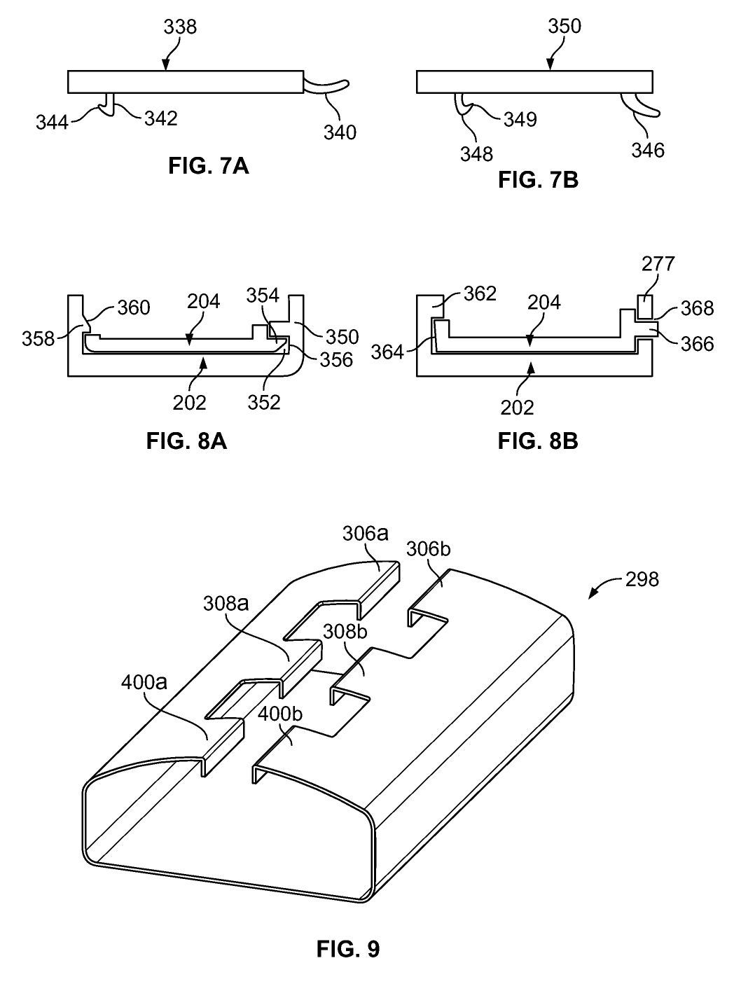

FIG. 7A and FIG. 7B schematically show embodiments in which tongue/receptive opening couplers are present at about the anterior end and posterior end of the device. FIG. 7A shows the lower cover 338 having a generally posterior extending curved tongue 340 at the posterior end and a downwardly extending tongue 342 about the anterior portion of the lower cover of the electronic module. “Downwardly” means oriented in the downward direction in the assembled device. The downwardly extending tongue 342 can have a small hook 344 but does not have any significant anterior extending portion, in that the anterior extending portion, if any, is small and can be push through the receptive opening of the inner upper portion of the reservoir module (not shown in FIG. 7A) with a force that has no anterior pointing force component (as in first threading the tongue through the opening towards the anterior, i.e., away from the other end of the device). Of course, the hook 344 can be provided to face the posterior or laterally if desired. FIG. 7B shows another embodiment has a curved tongue 346 extending about the posterior end and another curved tongue 348 about the anterior end from the bottom side of the lower cover 350 of the electronic module. The tongue 348 about the anterior end has a hook 351 for locking the lower cover 350 of the electronic module with the reservoir module. Both tongues 346, 348 generally curve towards the posterior so that the lower cover 350 can be inserted into their corresponding openings with a rolling motion having a vector facing the posterior. With tongues about at least one end of a module as described above, the electrotransport can be assembled readily, even entirely by feel if necessary. Although the couplers described above for securing the ends (i.e., the anterior end in FIG. 2) that last come together by the pivotal closing motion are preferably permanent couplers that once firmly pressed together would permanently lock the electronic module with the reservoir module, it is contemplated that disengageable couplers (e.g., snap button type couplers with a bulb insertable into a receptor socket or hole, or an insert against a springy release latch) can also be used. Devices in which the modules are permanently coupled provide an advantage that the unit is sturdy and the internal electronics and other features are well protected from mechanical disturbance or chemical intrusion and to provide secure electrical and mechanical engagement. Devices in which the modules are separable after use provides the advantage of reusing the electronic module.

FIG. 8A schematically shows an embodiment in which the reservoir module 202 has a foot 350 at which an opening 352 into which the tongue 354 of the electronic module 204 is inserted ends at a dead-end wall 356, so that the tongue 354 will rest on the dead-end wall as the electronic module 204 pivots about the posterior end to further engage the reservoir module 202 at the anterior end. Thus the opening 352 is part of a cavity or slot into which the tongue 352 extends. About the anterior end the reservoir module 202 has a catch 358 past which the electronic module 204 can have a member that slides or click-fits to engage firmly. In this embodiment, the catch 358 on the reservoir module 202 has a slope 360 on which a member about the anterior end of the electronic module can slide past to latch into the reservoir module 202.

FIG. 8B schematically shows an embodiment in which the anterior end of the reservoir module 202 has a catch 362 past which the anterior end of the electronic module 204 can have a member that slides or click-fits to engage firmly. In this embodiment, the anterior end of a member at the electronic module 204 has a slope 364 that can slide past the catch 362 to latch into the reservoir module 202. About the posterior end, the tongue 366 (e.g., at the lower cover 220 of the electronic module) extends into opening 368 of foot 277 (e.g., at inner upper portion 248 of the reservoir module) to enable pivotal motion.

It is noted that in many of the embodiments described herein, the tongue is positioned about the posterior end of the lower cover 220 of the electronic module 204 and the receptor for receiving the tongue is positioned about the posterior end of the inner upper portion 248 in reservoir module 202, it is to be understood that the tongue can be affixed to a different layer in the electronic module and the receptor can be positioned in a different layer in the reservoir module.

It is to be understood that although in the above embodiments the disengageable tongue or loosely engageable tongue is positioned at the posterior end of the electronic module and the opening to receive the tongue is positioned at the posterior end of the reservoir module, a person skilled in the art will be able to modify the above described embodiments to position the tongue and the receptive opening at the anterior end, or position the tongue on the reservoir module with the receptive opening on the electronic module. Further, although it is possible to have tongue/receptive opening couplers be present at both the anterior and posterior ends of the device, an alternative is that the tongue/receptive opening couplers be located about only one end of the electrotransport device or that the couplers are at least slightly different in size or shape so that the posterior end tongue would fit with the posterior end receptive opening, and vice versa.

For electrical communication, electrical connectors (couplers) 263, 265 can have a variety of sizes and shapes. The electrical connectors can have an insert or receptor that receives an insert. When two electrical connectors are to be coupled together, one electrical connector can have a generally female receptor shape for receiving a male insert and the other electrical couple can have the male insert. An electrical connector insert can include a bulb, button, hook, barb, post, slot U, etc., and can be inserted into receptors that have fingers, socket, grips, channel, etc. Further, electrical connectors can be pieces that match and can be biased together to contact for electrical communication. Other shapes of inserts and receptors are contemplated so long as they can be coupled together to provide electrical conduction when the electronic module and the reservoir module are pressed together. Other than metallic or alloy material, at least some of the electrical connectors can also be made with other conducting material such as carbon, conductive polymers, etc. Furthermore, electroplated or coated materials are also contemplated.

FIG. 9 shows an embodiment of an electrical connector that can receive an insert. The electrical connector 298 has a generally female receptor shape for receiving an insert. The electrical connector receptor 298 has fingers 306A, 308A, 400A pointing towards fingers 306B, 308B, 400B and vice versa. An electrical connector insert, which can include a bulb, hook, barb, post, etc., can be inserted between the fingers 306A, 308A, 400A, 306B, 308B, 400B to engage the electrical connector receptor 298 for electrical conduction. Another kind of insert is one that is bar-shaped or has a cross section of a slot U that can be press-fitted to insert into a channel-shaped receptor. Other shapes of inserts and receptors are contemplated so long as they can be coupled together to provide electrical conduction and optionally mechanical retaining engagement when the electronic module 204 and the reservoir module 202 are pressed together. Other than metallic or alloy material, at least some of the electrical connectors can also be made with other conducting material such as carbon, conductive polymers, etc. Furthermore, electroplated or coated materials are also contemplated. Of course, the connector 298 can also offer electrical contact with another electrical connector that simply presses on the face thereof. The resilient (or springy) nature of the connector 298 provides a force to bias the fingers 306A, 308A, 400A, 306B, 308B, 400B towards the other connector to maintain electrical contact. The other connector can also have features that bias toward connector 298, e.g., with a configuration similar to connector 298.

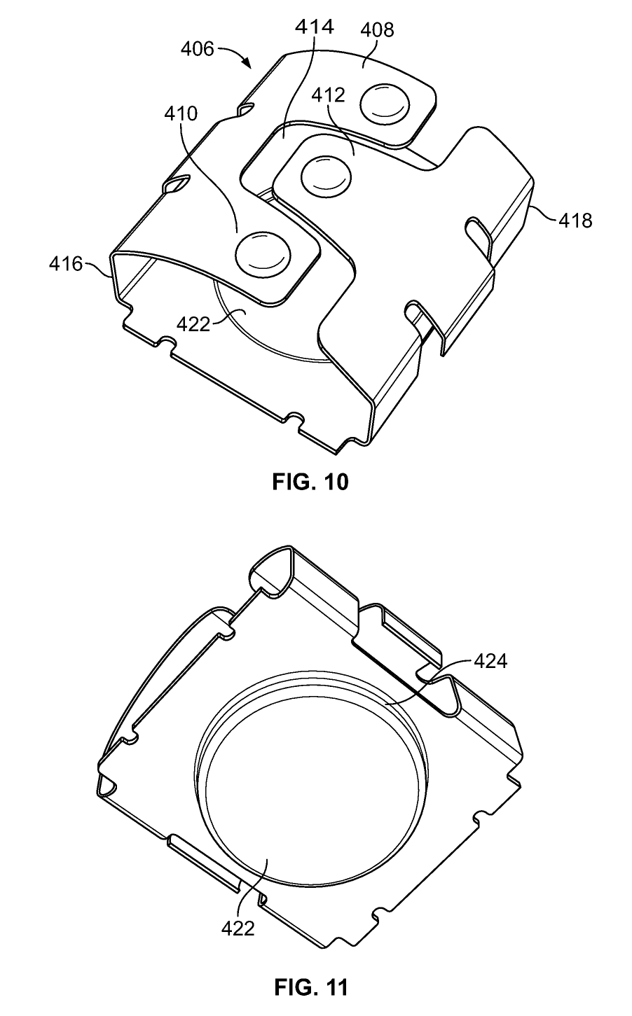

FIG. 10 shows a perspective top view of another embodiment of an electrical connector 406 having bent fingers 408, 410 on one side and finger 412 on an opposite side, each pointing to the other side. FIG. 11 shows a schematic perspective bottom view of the electrical connector receptor of FIG. 10. The fingers 408, 410, 412 are bent at an oblique direction so they have a vector component directing outward (i.e., toward another opposing electrical connector to couple therewith). The fingers 408, 410, 412 are made of a springy material (e.g., metal) that if pressed inward they provide a reacting biasing force outwards. The fingers 408, 410, 412 also are in an interlocking configuration as fingers of two hands interlock such that when pressed, the fingers come together to provide a contact surface with a decreasing gap 414 for better contact with the opposing electrical connector. Support extensions 416, 418 extend from two opposite sides of a bottom 420 to the fingers 408, 410, 412 to provide room for the fingers to flex. The bottom 420 has a foundation 422 extending further inward to anchor to the inner upper portion 248 of the reservoir module 202. The foundation 422 has a bend 424 from the bottom 420 to provide anchoring to the inner upper portion 248 either by mechanical force and/or by chemical adhesive or bonding.

Electrical connectors that provide electrical connection by biasing force rather than insertion are well suited for the present invention because the pivotal motion after first ends of the two modules are engaged allows a leverage to be used to easily press the electrical connectors together as the second ends of the modules are becoming engaged. The lever advantage allows the modules to come together, pressing down on the springy electrical connectors to result in a reacting biasing force that biases components (e.g., fingers) of one connector towards the opposite connectors. The biasing force allows the electrical connectors to remain in electrical contact without mechanical grasping or gripping such as those present in receptors for inserter connectors. Because of the biasing force, even if the parts modules are shaken as the device is being handled the electrical connection will remain intact. Further, electrical connectors that couple by insertion of an insert into a receptor are also suitable because the leverage advantage in pivotal motion also can be used to force an insert into a tight receptor.

FIG. 6A shows a top plan view of the embodiment of FIG. 2 after full assembly. FIG. 6B shows an embodiment in which a digital display 237 is present. Preferably, the upper layer 228 in the electronic module 204 and the outer upper portion 252 in the reservoir module 202 are both made with the same resilient material so that when the electronic module 204 and the reservoir module 202 are fitted together they form a unit that looks as if it is made of the same material. This provides assurance that the two modules were oriented properly and that assembly is correctly done. Of course, if desired, alternative designs can be done such that the module orientation by visual inspection is not important, for example, where one or both of the modules have ends that look similar.

The reservoir of the electrotransport delivery devices generally can contain a gel matrix, with the drug solution uniformly dispersed in at least one of the reservoirs. Obviously, other types of reservoirs such as membrane-confined reservoirs are possible and contemplated. The application of the present invention is not limited by the type of reservoir used. Gel reservoirs are described, e.g., in U.S. Pat. Nos. 6,039,977 and 6,181,963, which are incorporated by reference herein in their entireties. Suitable polymers for the gel matrix can comprise essentially any synthetic and/or naturally occurring polymeric materials suitable for making gels. A polar nature is preferred when the active agent is polar and/or capable of ionization, so as to enhance agent solubility. Optionally, the gel matrix can be water swellable nonionic material. Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent.

Incorporation of the drug solution into the gel matrix in a reservoir can be done in any number of ways, i.e., by imbibing the solution into the reservoir matrix, by admixing the drug solution with the matrix material prior to hydrogel formation, or the like. In additional embodiments, the drug reservoir may optionally contain additional components, such as additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Such materials can be included by on skilled in the art.

The drug reservoir can be formed of any material as known in the prior art suitable for making drug reservoirs. The reservoir formulation for transdermally delivering cationic drugs by electrotransport is preferably composed of an aqueous solution of a water-soluble salt, such as HCl or citrate salts of a cationic drug, such as fentanyl or sufentanil. More preferably, the aqueous solution is contained within a hydrophilic polymer matrix such as a hydrogel matrix. The drug salt is preferably present in an amount sufficient to deliver an effective dose by electrotransport over a delivery period of up to about 20 minutes, to achieve a systemic effect. The drug salt typically includes about 0.05 to 20 wt % of the donor reservoir formulation (including the weight of the polymeric matrix) on a fully hydrated basis, and more preferably about 0.1 to 10 wt % of the donor reservoir formulation on a fully hydrated basis. In one embodiment the drug reservoir formulation includes at least 30 wt % water during transdermal delivery of the drug. Delivery of fentanyl and sufentanil has been described in U.S. Pat. No. 6,171,294, which is incorporated by reference herein. The parameter such as concentration, rate, current, etc. as described in U.S. Pat. No. 6,171,294 can be similarly employed here, since the electronics and reservoirs of the present invention can be made to be substantially similar to those in U.S. Pat. No. 6,171,294.

The drug reservoir containing hydrogel can suitably be made of any number of materials but preferably is composed of a hydrophilic polymeric material, preferably one that is polar in nature so as to enhance the drug stability. Suitable polar polymers for the hydrogel matrix include a variety of synthetic and naturally occurring polymeric materials. A preferred hydrogel formulation contains a suitable hydrophilic polymer, a buffer, a humectant, a thickener, water and a water soluble drug salt (e.g. HCl salt of an cationic drug). A preferred hydrophilic polymer matrix is polyvinyl alcohol such as a washed and fully hydrolyzed polyvinyl alcohol (PVOH), e.g. Mowiol 66-100 commercially available from Hoechst Aktiengesellschaft. A suitable buffer is an ion exchange resin which is a copolymer of methacrylic acid and divinylbenzene in both an acid and salt form. One example of such a buffer is a mixture of POLACRILIN (the copolymer of methacrylic acid and divinyl benzene available from Rohm & Haas, Philadelphia, Pa.) and the potassium salt thereof. A mixture of the acid and potassium salt forms of POLACRILIN functions as a polymeric buffer to adjust the pH of the hydrogel to about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g. METHOCEL K100MP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments can be about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise. In certain preferred embodiments, the drug-containing hydrogel formulation includes about 10 to 15 wt % polyvinyl alcohol, 0.1 to 0.4 wt % resin buffer, and about 1 to 30 wt %, preferably 1 to 2 wt % drug. The remainder is water and ingredients such as humectants, thickeners, etc. The polyvinyl alcohol (PVOH)-based hydrogel formulation is prepared by mixing all materials, including the drug, in a single vessel at elevated temperatures of about 90 degree C. to 95 degree C. for at least about 0.5 hour. The hot mix is then poured into foam molds and stored at freezing temperature of about −35 degree C. overnight to cross-link the PVOH. Upon warming to ambient temperature, a tough elastomeric gel is obtained suitable for ionic drug electrotransport.

A variety of drugs can be delivered by electrotransport devices. In certain embodiments, the drug is a narcotic analgesic agent and is preferably selected from the group consisting of fentanyl and related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine.

Some ionic drugs are polypeptides, proteins, hormones, or derivatives, analogs, mimics thereof. For example, insulin or mimics are ionic drugs that can be driven by electrical force in electrotransport.

For more effective delivery by electrotransport salts of certain pharmaceutical agents are preferably included in the drug reservoir. Suitable salts of cationic drugs, such as narcotic analgesic agents, include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. The more preferred salt is chloride.

A counterion is present in the drug reservoir in amounts necessary to neutralize the positive charge present on the cationic drug, e.g. narcotic analgesic agent, at the pH of the formulation. Excess of counterion (as the free acid or as a salt) can be added to the reservoir in order to control pH and to provide adequate buffering capacity. In one embodiment of the invention, the drug reservoir includes at least one buffer for controlling the pH in the drug reservoir. Suitable buffering systems are known in the art.

Obviously, the present invention is also applicable where the drug is an anionic drug. In this case, the drug is held in the cathodic reservoir (the negative pole) and the anoidic reservoir would hold the counterion. A number of drugs are anionic, such as cromolyn (antiasthmatic), indomethacin (anti-inflammatory), ketoprofen (anti-inflammatory) and ketorolac tromethamine (NSAID and analgesic activity), and certain biologics such as certain protein or polypeptides.

### Method of Making

A device according to the present invention can be made by forming the layers separately and assembling the layers into the electronic module and the reservoir module. The polymeric layers can be made by molding. Some of the layers can be applied together and secured. Some of the layers can be comolded, for example, by molding a second layer onto a first layer. For example, the upper layer and lower layer of the upper cover (or top cover) can be comolded together. Some of the layers can be affixed together by adhesive bonding or mechanical anchoring. Such chemical adhesive bonding methods and mechanical anchoring methods are known in the art. As described before, once the electronic module and the reservoir module are formed, they can be packaged separately. Before use, the two modules can be removed from their respective packages and assembled to form the device for electrotransport. The device can then be applied to the body surface by adhesion.

The above-described exemplary embodiments are intended to be illustrative in all respects, rather than restrictive, of the present invention. Thus the present invention is capable of many variations in detailed implementation that can be derived from the description contained herein by a person skilled in the art, e.g., by permutation or combination of various features. All such variations and modifications are considered to be within the scope of the present invention. Although iontophoretic devices are described in detail as illustration for showing how an electronic module and an agent module are coupled and work together, a person skilled in the art will know that electronic module and agent module in other electrotransport devices can be similarly coupled and work together. The entire disclosure of each patent, patent application, and publication cited or described in this document is hereby incorporated herein by reference.

We claim:

1. An electrotransport device for delivering a therapeutic agent through a body surface of a patient, the device comprising:
agent module having a first end, a second end, and including a compartment that contains the therapeutic agent for electrotransporting through the body surface, the agent module including first agent module (AM) coupler about the first end; and
electronic module having a first end and a second end corresponding to the first end and second end of the agent module, the electronic module having first electronic module (EM) coupler about the first end of the electronic module for coupling with the first AM coupler such that as the first AM coupler matingly engages with the first EM coupler the electronic module and the agent module can be pressed together pivoting about where the first AM coupler engages the first EM coupler, the electronic module including circuitry for electrically driving the therapeutic agent for electrotransport.

2. The device of claim 1, wherein the agent module is a reservoir module and the reservoir module has a reservoir as the compartment and has a second AM coupler, the electronic module has a second EM coupler for retaining the second AM coupler in coupling, and wherein one of the first AM coupler and the first EM coupler is a tongue and the other of the first AM coupler and the first EM coupler having an opening such that the tongue is inserted into the opening thereby allowing the reservoir module and the electronic module to come together in a pivoting motion.

3. The device of claim 2, wherein the tongue is located about the first end of either the agent module or the electronic module and extends in a direction that has a directional component in the direction from the second end toward the first end of the module on which the tongue is located.

4. The device of claim 2, wherein the tongue is located about the first end of either the agent module or the electronic module and extends in a direction that has a directional component in the direction from the second end toward the first end of the module on which the tongue is located and wherein the tongue has a curve or angled portion such that the curve or angled portion slides against an edge of an opening of the other of the electronic module and the agent module when said modules are pressed together in pivoting motion.

5. The device of claim 2, wherein the tongue is located about the first end of the electronic module and extends substantially toward the direction of the first end from the second end of the electronic module and wherein the tongue has a curve or angled portion such that the curve or angled portion slides against an edge of an opening of the other of the electronic module and the agent module when said modules are pressed together in pivoting motion.

6. The device of claim 2, wherein the agent module including a cutout forming a channel with channel walls, the electronic module having agent module (AM)—facing lower cover that fits inside the channel and can matingly stack onto an electronic module (EM)-facing layer in the agent module.

7. The device of claim 2, wherein the electronic module includes a printed circuit board (PCB) having circuitry for controlling function of the device, an upper cover and an AM-facing lower cover protecting the PCB in the middle.

8. The device of claim 2, wherein the electronic module includes a printed circuit board (PCB) having circuitry for controlling function of the device, an upper cover and an AM-facing lower cover protecting the PCB in the middle and wherein electrical connectors of the PCB for connecting with the agent module are either not covered by the lower cover or connected to conductive pads in the lower cover.

9. The device of claim 2, wherein the electronic module includes a printed circuit board (PCB) having circuitry for controlling function of the device, an upper cover and an AM-facing lower cover protecting the PCB in the middle and wherein the tongue is part of the AM-facing lower cover and located about the first end thereof.

10. The device of claim 2, wherein the tongue is part of an AM-facing lower cover of the electronic module and located about the first end thereof and the agent module includes a rigid EM-facing layer supporting electrical connectors for connecting with electrical connectors from the electronic module, said rigid EM-facing layer having the opening about the first end of the agent module for receiving the tongue from the electronic module.

11. The device of claim 2, wherein the electronic module includes a printed circuit board (PCB) having circuitry for controlling function of the device, an upper cover and an AM-facing lower cover protecting the PCB in the middle, the upper cover having a polymeric material less rigid than the material of the AM-facing lower cover such that the upper cover can match the agent module to be liquid resistant.

12. The device of claim 2, wherein the agent module includes a rigid EM-facing layer having the opening for receiving the tongue about the first end of the agent module, the tongue is part of an AM-facing lower cover of the electronic module and located about the first end thereof, the tongue has a major directional component extending toward the direction of the first end of the electronic module and wherein the tongue has a curve or angled portion such that the curve or angled portion slides in the opening when the electronic module and the agent module are pressed together in pivoting motion.

13. The device of claim 2, wherein the second couplers of the electrical module and the agent module can be permanently engaged by snap-fitting together.

14. The device of claim 2 wherein the second couplers of the electrical module and the agent module are selected from barb and receptor, bulb and receptor, angled ledge and receptor, and press-fit slot insert and channel.

15. A method of making an electrotransport device for delivering a therapeutic agent through a body surface of a patient, comprising matingly engaging an insert at one end of one of an agent module and an electronic module to a receptor at the other of said modules and pressing the modules together by pivoting about where the modules matingly engage, the agent module containing a compartment including the therapeutic agent.

16. The method of claim 15, wherein the agent module has a first end and a second end, and including one or more reservoirs one of which is the compartment that contains the therapeutic agent for delivery through the body surface, the agent module including first agent module (AM) coupler about the first end, the electronic module having a first end and a second end corresponding to the first end and second end of the agent module, the electronic module including circuitry for electrically driving the therapeutic agent for electrotransport and having first electronic module (EM) coupler about the first end of the electronic module for coupling with the first AM coupler, including matingly engaging a tongue from one of the first AM coupler and the first EM coupler with an opening of the other of said couplers and pressing the electronic module and the agent module together pivoting about where the first AM coupler engages the first EM coupler.

17. The method of claim 16, comprising inserting a tongue located about the first end of either the agent module or the electronic module and which extends in a direction that has a directional component in the direction from the second end towards the first end of the module on which the tongue is located into an opening in the other of the agent module and the electronic module, the tongue having a curve or angled portion, the method further comprising sliding the curve or angled portion against an edge of the opening to move the electronic module and the agent module closer together in pivoting motion.

18. The method of claim 16, wherein the tongue is located about the first end of the electronic module and extends with a directional major component toward the direction of the first end from the second end of the electronic module and wherein the tongue has a curve or angled portion such that the curve or angled portion slide against an edge of an opening of the other of the electronic module and the agent module when said modules are pressed together in pivoting motion.

19. The method of claim 16, comprising forming in the agent module a cutout as a channel with channel walls, including in the electronic module an agent module (AM)-facing lower cover that fits inside the channel and can matingly stack onto a electronic module (EM)-facing layer in the agent module, including pivotally pressing the agent module and the electronic module together to move the AM-facing lower cover into the channel and matingly stack the AM-facing lower cover onto the EM-facing layer.

20. The method of claim 16, comprising including in the electronic module a printed circuit board (PCB) having circuitry for controlling function of the device, an upper cover and an AM-facing lower cover protecting the PCB in the middle, the upper cover having a polymeric material less rigid than the material of the AM-facing lower cover, further comprising fitting the upper cover with the agent module to be liquid resistant.

21. An electrotransport device for delivering a therapeutic agent through a body surface of a patient, the device comprising:
reservoir module having a first end, a second end, and including one or more reservoirs one of which contains the therapeutic agent for electrotransporting through the body surface, the reservoir module including a slot about the first end; and
electronic module having a first end and a second end corresponding to the first end and second end of the reservoir module, the electronic module including a tongue having a curve or angled portion about the first end for matingly engaging with the slot in the reservoir module such that the curve or angled portion slides on an edge of the slot as the electronic module and the reservoir module are pressed together to result in pivoting motion about where the tongue matingly engages the slot, the electronic module including circuitry for electrically driving the therapeutic agent for electrotransport.

22. A method of using an electrotransport device for delivering a therapeutic agent through a body surface of a patient, comprising matingly engaging an insert at one end of one of a reservoir module and an electronic module to a receptor at the other of said modules so that the modules are free to pivot and pressing the modules together by pivoting about where the modules matingly engage and applying the resulting device on a body surface of a patient for electrotransport, wherein the reservoir module containing a reservoir including the therapeutic agent.
